(19) United States
(12) Patent Application Publication
(10) Pub. No.: US 2021/0222035 A1
(43) Pub. Date: Jul. 22, 2021

(54) BIOCOMPATIBLE ADHESIVE MATERIALS WITH IONIC AND COVALENT INTERACTION

(71) Applicant: BioDevek, Inc., Cambridge, MA (US)

(72) Inventors: Gonzalo Munoz Taboada, Coruna (ES); Natalie Artzi, Brookline, MA (US); Elazer R. Edlman, Brookline, MA (US)

(21) Appl. No.: 17/055,856

(22) PCT Filed: May 15, 2019
(86) PCT No.: PCT/US19/32458

§ 371 Date: Nov. 16, 2020

Related U.S. Application Data
(60) Provisional application No. 62/672,144, filed on May 16, 2018.
(60) Provisional application No. 62/672,046, filed on May 15, 2018.

(51) Int. Cl. C09J 105/02; A61L 24/04; A61L 24/00

(52) U.S. Cl. CPC C09J 105/02; A61L 24/0015; A61L 24/001; A61L 24/043

(57) ABSTRACT

Disclosed are biocompatible adhesive materials for adhering, sealing, or treating one or more biological tissues. Generally, the biocompatible adhesive materials comprise a dendrimer component and one or more polymer components. These biocompatible adhesive materials are useful as tissue adhesives, tissue sealants, tissue treatments, matrix materials, fillers, coatings, or a combination thereof.

### RELATED APPLICATIONS

This application claims the benefit of priority to U.S. Provisional Application No. 62/672,144, filed May 16, 2018; and U.S. Provisional Application No. 62/672,046, filed May 15, 2018.

### BACKGROUND

Sealants, adhesives and mechanical barriers play an important role in helping patients recover from surgery or trauma. Sealants, adhesives and mechanical barriers are useful in treating patients suffering from a variety of in vivo (e.g., internal) or topical conditions, including lacerations, tears, wounds, ulcers, anastamoses, and surgical procedures. Sealants or adhesives can generally be used in any indication or application for which a suture or staple is presently used, and the sealant or adhesive often provides a better outcome than a suture or staple. Sealants or adhesives can also be applied more quickly to the injury site and often provide a better seal over the wound and healing.

A number of tissue adhesives have been used in various medical procedures and applications, including topical wound closure, supplementing or replacing surgical sutures or staples, adhesion of synthetic materials to biological tissues, and drug delivery. A number of known tissue adhesives, however, are unsuitable for many applications, for example, due to toxic degradation products, slow curing, poor mechanical strength, and other drawbacks.

Several varieties of hydrogel adhesives have been developed, which are nontoxic and have improved properties. These hydrogels are generally formed by reacting a component having nucleophilic groups with a component having electrophilic groups that react to form a cross-linked network. However, these hydrogels typically dissolve too quickly, lack sufficient adhesion, or have insufficient mechanical strength.

Therefore, it would be desirable to provide improved adhesive formulations that overcome one or more of the above-described disadvantages.

### SUMMARY

In one aspect, compositions are provided for adhering, sealing, or treating one or more biological tissues. The adhesive material composition comprises one or more polymer components and a dendrimer component. In certain embodiments, the polymer components comprise polymers having one or more aldehyde groups. In certain embodiments, the dendrimer component comprises a dendrimer having at least two arms or branches with one or more surface groups. In certain embodiments, the dendrimer component comprises dendrimer having amine group on the surface.

### BRIEF DESCRIPTION OF THE DRAWINGS

FIG. 1 depicts sample fixation.

FIG. 2 depicts the experimental setup for burst pressure measurements.

FIG. 3 depicts the effect of the dextran component in the burst pressure test following an adapted ASTM standard test method for burst strength of surgical sealants.

FIG. 4 depicts the effect of the alginate component in the burst pressure test following an adapted ASTM standard test method for burst strength of surgical sealants.

FIG. 5 depicts the effect of the dendrimer component in the burst pressure test following an adapted ASTM standard test method for burst strength of surgical sealants.

FIG. 6 depicts the effect of different percentages of dextran and alginate with 30% dendrimer at pH 10 in the burst pressure test following an adapted ASTM standard test method for burst strength of surgical sealants (spray applicator).

FIG. 7 depicts the effect of different percentages of dextran and alginate with 30% dendrimer at pH 9.3 in the burst pressure test following an adapted ASTM standard test method for burst strength of surgical sealants (syringe applicator).

FIG. 8 depicts the effect of different percentages of dextran and alginate with 30% dendrimer at pH 10 in the burst pressure test following an adapted ASTM standard test method for burst strength of surgical sealants (syringe applicator).

FIG. 9 depicts Neutral Red Uptake (NRU) cell viability data at pH 10 for various dextran-alginate-dendrimer compositions (Dex-Alg-Den).

FIG. 10 depicts the effect of different percentages of dextran and alginate with 30% dendrimer at pH 9.3 in the cell viability measured by the NRU standard method.

FIG. 11 depicts the effect of different percentages of dextran and alginate with 30% dendrimer at pH 10 in the cell viability measured by the NRU standard method.

FIG. 12 depicts a double-barrel syringe charged with: (i) 30 wt % Dendrimer in water; and (ii) 15 wt % oxidized dextran and 10 wt % oxidized alginate in water.

### DETAILED DESCRIPTION

The present disclosure relates, at least in part, to a biocompatible adhesive material for

use with biological tissues and/or medical implants. The biocompatible adhesive material includes a dendrimer component and one or more linear or branched polymer components. The dendrimer and one or more two linear or branched polymer components are designed to achieve new and non-straightforward properties. Certain information related to certain components, linear or branched polymers, dendrimers, and methods that may be useful in the context of certain embodiments of the present disclosure is disclosed in U.S. Pat. No. 10 8,802,072, which is incorporated herein by reference in its entirety for all purposes.

The one or more linear or branched polymer components are two different polymers (e.g., dextran-aldehyde and alginate-aldehyde) that are mixed together to form a mixture. The two linear or branched polymer components provide synergistic properties that enhance both the internal cohesive properties and the adhesive properties with biological tissue/synthetic grafts. Alginate is oxidized to generate aldehyde groups on its backbone that will covalently react with non-protonated dendrimer amines. The modified alginate backbone remains negatively charged, creating an additional mechanism of interaction, both with the tissue and with the positively charged dendrimer component. While modified dextran modulates material degradation rate, modified alginate plays a significant role in enhancing the adhesion and participating in mechanical energy absorption. Alginate is an anionic polysaccharide, which is oxidized for its use as adhesion enhancer.

The dendrimer component has primary amine surface groups. The pH of the dendrimer

component is tuned to modify the ratio of protonated/unprotonated amines. By decreasing the
pH of the dendrimer, a higher ratio of protonated to unprotonated amines is achieved. Positively charged protonated amines can interact with the negatively charged backbone of the new polymer component (COO— of the modified alginate), thus toughening the material. These positively charged amines will also interact with negatively charged groups present on biological tissue or synthetic grafts, promoting adhesion capabilities.

In one aspect, provide herein are biocompatible adhesive materials, comprising a dendrimer component and a linear or branched polymer component.

In certain embodiments, the dendrimer has a molecular weight of about 1,000 to about 1,000,000 Da. In certain embodiments, the dendrimer has a molecular weight of about 5,000 to about 500,000 Da. In certain embodiments, the dendrimer has a molecular weight of about 10,000 to about 100,000 Da.

In another certain embodiment, the dendrimer is a generation dendrimer having primary amines on at least 25% of the dendrimer's surface groups. In another certain embodiment, the dendrimer is a generation dendrimer having primary amines on at least 50% of the dendrimer's surface groups. In another certain embodiment, the dendrimer is a generation dendrimer having primary amines on at least 75% of the dendrimer's surface groups.

In certain embodiments, the dendrimer extends through at least 2-6 generations. In certain embodiment, the dendrimer extends through at least 3-5 generations.

In certain embodiments, the dendrimer is a generation 5 PAMAM-derived dendrimer.

In certain embodiments, the linear or branched polymer is an anionic polymer. In certain embodiments, the linear or branched polymer is a polysaccharide. In certain embodiments, the linear or branched polymer is an alginate.

In certain embodiments, the linear or branched polymer has a molecular weight from about 3,000 to about 3,000,000 Da. In certain embodiments, the linear or branched polymer has a molecular weight from about 10,000 to about 1,000,000 Da. In certain embodiments, the linear or branched polymer has a molecular weight from about 30,000 to about 300,000 Da.

In certain embodiments, the biocompatible adhesive material further comprising a second linear or branched polymer component.

In certain embodiments, he second linear or branched polymer is an oxidized polysaccharide. In certain embodiments, the second linear or branched polymer is an oxidized dextran.

In certain embodiments, at least 10% of the dextran's primary hydroxyls are oxidized to aldehydes. In certain embodiments, at least 25% of the dextran's primary hydroxyls are oxidized to aldehydes. In certain embodiments, at least 40% of the dextran's primary hydroxyls are oxidized to aldehydes.

In certain embodiments, the dendrimer component, the linear or branched polymer component, or the second linear or branched polymer component further comprises an additive selected from the group consisting of foaming agents, pH modifiers, thickeners, antimicrobial agents, colorants, surfactants, radio-opaque agents, and biologically active components

In certain embodiment, the dendrimer component, the linear or branched polymer component, or the second linear or branched polymer component is in a solution. In certain embodiment, the dendrimer component, the linear or branched polymer component, or the second linear or branched polymer component is in an aqueous solution.

In certain embodiments, the aqueous solution of the dendrimer component, the linear or branched polymer component, or the second linear or branched polymer component has a pH from about 7 to about 11. In certain embodiments, the aqueous solution of the dendrimer component, the linear or branched polymer component, or the second linear or branched polymer component has a pH from about 8 to about 10. In certain embodiments, the aqueous solution of the dendrimer component, the linear or branched polymer component, or the second linear or branched polymer component has a pH about 9.

In certain embodiments, the dendrimer component constitutes 1-50 wt % of the aqueous solution. In certain embodiments, the dendrimer component constitutes 10-40 wt % of the aqueous solution. In certain embodiments, the dendrimer component constitutes about 30 wt % of the aqueous solution.

In certain embodiments, the linear or branched polymer component constitutes 1-30 wt % of the aqueous solution. In certain embodiments, the linear or branched polymer component constitutes 10-25 wt % of the aqueous solution. In certain embodiments, the linear or branched polymer component constitutes about 20 wt % of the aqueous solution.

In certain embodiments, the second linear or branched polymer component constitutes 1-30 wt % of the aqueous solution. In certain embodiments, the second linear or branched polymer component constitutes 10-25 wt % of the aqueous solution. In certain embodiments, the second linear or branched polymer component constitutes about 20 wt % of the aqueous solution.

In another aspect, provided are methods of adhering, sealing, or treating one or more biological tissues or prosthetic materials, comprising applying to one or more surfaces of said one or more biological tissues the biocompatible adhesive material.

39. In certain embodiments, the biocompatible adhesive material is applied as a spray. In certain embodiments, the biocompatible adhesive material is applied via a syringe.

### Exemplary Medical Applications

### Skin Lacerations

Skin lacerations are tears in the skin produced by accidents, trauma, or as a result of a surgical procedure. Lacerations often require treatment in order to close the hole in the skin, stop bleeding, and prevent infection. Minor lacerations in the skin may be treated using an adhesive tissue to cover the wound. However, larger laceractions often require sutures or a glue to help seal the wound. For example, it is generally recommended that sutures or a glue be used to treat lacerations deeper than 0.25 inches having a jagged edge or loose flap of tissue. The location of the laceration may also affect the form of treatment. For example, it is advantageous to treat a skin laceration on a joint using a glue because an adhesive bandage tends to limit mobility of the joint. The use of sutures or glues to treat skin lacerations can also reduce the chance of scar formation.

### Liver Lacerations

Lacerations of the liver can occur from trauma or as a result of a surgical procedure. The liver is a highly vascularized organ and bleeds profusely when lacerated or traumatized. Liver lacerations are difficult to repair owing to the nature of liver tissue. Liver tissue has very weak cohesive strength, and, consequently, sutures and staples are not satisfactory because they may pull through the liver tissue. The lack of satisfactory wound treatment methods for liver lacerations combined with the fact that it is difficult to reach the arteries that feed the liver renders liver lacerations particularly serious. In fact, severe lacerations of the liver often result in the patient's death due to bleeding. Thus, new materials to treat liver lacerations are needed.

### Lung Surgery

The sealants and methods of the present invention are useful in lung surgery. Types of lung surgery include lobectomy, lung biopsy, lung-tissue removal, and pneumonectomy. Risks associated with lung surgery include wound infection; post-surgical internal bleeding; air leaks; pain or numbness at the incision site; and infection of the lungs (pneumonia). Further, air leakage is frequently observed after thoracic procedures, such as pulmonary resection and decortication. It is important to create an air-tight seal so as to prevent or reduce severe complications, such as bronchopleural fistulas and infection resulting from extended chest tube drainage, extended recovery time, and postoperative morbidity related to pulmonary surgery. The sealants and methods of the invention should decrease or eliminate some of the problematic aspects of lung surgery, such as treatment of pneumothorax and pulmonary leaks.

### Cornea—Corneal Lacerations/Perforations

Corneal perforations are produced by a variety of medical conditions (e.g., infection, inflammation, xerosis, neurotrophication, and degeneration) and traumas (chemical, thermal, surgical, and penetrating). Unfortunately, corneal perforations often lead to loss of vision and a decrease in an individual's quality of life. Depending on the type and the origin of the perforation, different treatments may be effective, ranging from suturing the wound to a cornea graft. However, the surgical procedures are difficult given the delicate composition of the cornea and the severity of the wound which increase the likelihood for leakage and severe astigmatism after surgery. In certain cases, for example, perforations that cannot be treated by standard suture procedures, tissue adhesives (glues) are used to repair the wound. This type of treatment is very attractive because the method is simple, quick and safe, and corresponds to the requirement of a quick restoration of the integrity of the globe, avoiding further complications. Besides an easy and fast application on the wound, the characteristics of an adhesive include: 1) bind to the tissue (necrosed or not, very often wet) with an adequate adhesion force; 2) be non-toxic; 3) be biodegradable or resorbable; 4) be sterilizable; and 5) not interfere with the healing process.

Various alkyl-cyanoacrylates are available for the repair of small perforations. However, these “super glues” present major inconveniences. Their monomers, in particular those with short alkyl chains, can be toxic, in part due to their ability to produce formaldehyde in situ. They also polymerize too quickly leading to applications that might be difficult and, once polymerized, the surface of the glue is rough and hard which leads to patient discomfort and a need to wear contact lens. Even though cyanoacrylate is tolerated as a corneal sealant, a number of complications have been reported including cataract formation, corneal infiltration, glaucoma, giant papillary conjunctivitis, and symblepharon formation. Furthermore, in more than 60% of the patients, additional surgical intervention is needed.

Other glues have also been developed. Adhesive hemostats, based on fibrin, are usually constituted of fibrinogen, thrombin and factor XIII. Systems with fibrinogen and photosensitizers activated with light are also being tested. If adhesive hemostats have intrinsic properties which meet the requirements for a tissue adhesive, then autologous products (time consuming in an emergency) or severe treatments before clinical use are needed to avoid any contamination to the patient. An ideal sealant for corneal perforations should 1) not impair normal vision, 2) quickly restore the intraocular pressure (IOP), 3) maintain the structural integrity of the eye, 4) promote healing, 5) adhere to moist tissue surfaces, 6) possess solute diffusion properties which are molecular weight dependent and favorable for normal cornea function, 7) possess rheological properties that allow for controlled placement of the polymer on the wound, and 8) polymerize under mild conditions.

The use of sutures has limitations and drawbacks. First, suture placement itself inflicts trauma to corneal tissues, especially when multiple passes are needed. Secondly, although suture material has improved, sutures such as 10-0 nylon (which is the suture of choice in the cornea and elsewhere) can act as a nidus for infection and incite corneal inflammation and vascularization. With persistent inflammation and vascularization, the propensity for corneal scarring increases. Thirdly, corneal suturing often yields uneven healing and resultant regular and irregular astigmatism. Postoperatively, sutures are also prone to becoming loose and/or broken and require additional attention for prompt removal. Finally, effective suturing necessitates an acquired technical skill that can vary widely from surgeon to surgeon and can also involve prolonged operative time.

### Cornea—Corneal Transplants

During a corneal transplant or penetrating keratoplasty surgery the diseased cornea is removed with a special round cutting tool called a trephine. The donor cornea is cut to a matching size. Then, the donor cornea is placed upon the eye and secured in place with approximately 16 sutures around the transplant to secure the new cornea in place. A sutureless procedure would be highly desirable because sutures are associated with the following drawbacks and others: (1) sutures provide a site for infection, (2) the sutured cornea takes 3 months to heal before the sutures need to be removed, and (3) the strain applied to the new cornea tissue from the sutures can distort the cornea. An ocular adhesive may also serve as an adjuvant to sutures and/or reduce the necessary number of sutures.

### Cornea—Clear Corneal Incision

Clear corneal incisions in the temporal cornea offer several advantages with phacoemulsification. The major advantage associated with phacoemulsification is the reduction in size of the entrance wound. Smaller wounds require fewer sutures or even no sutures at all, minimizing induction of astigmatism, decreasing bleeding and subconjunctival hemorrhage, and speeding the recovery of visual acuity. See Agapitos, P. J. Curr. Opin. Ophthalmol. 1993, 4, 39-43 and Lyle, W. A.; Jin, G. J. J. Cataract Refract. Surg. 1996, 22, 1456-1460. Surgeons typically examine the clear corneal incisions at the completion of the procedure by inflating the anterior chamber with balanced salt solution and applying pressure to the anterior cornea to check for leakage from the wound. If there is some leakage, the wound may be hydrated with balanced saline solution to seal fully the wound. This is done by injecting balanced saline solution into the open stromal edges. Hydration forces the two edges of the wound together, creating a tight seal. The endothelial cell pump can then remove the fluid from both the anterior and posterior portions of the wound, further sealing the wound together. See Fine, I. H. J. Cataract Refract. Surg. 1991, 17 (Suppl), 672-676. These tests for fluid flow, however, make several assumptions, including that the eye will remain well pressurized during the early postoperative period, that the hydrated wound will not be rapidly deturgesced by the corneal endothelium, and that the absence of aqueous outflow from the wound correlates with the inability of surface fluid from the tear film to flow into the wound, possibly contaminating the aqueous humor and predisposing to infection. However, intraocular pressure is known to vary in the postoperative period, frequently dropping to less than 5 mm Hg, and telemetric intraocular pressure monitoring devices suggest that large fluctuations in intraocular pressure occur in individual eyes in response to blinking. See Shingleton, B. J.; Wadhwani, R. A.; O'Donoghue, M. W.; Baylus, S.; Hoey, H. J. Cataract Refract. Surg. 2001, 27, 524-527 and Percicot, C. L.; Schnell, C. R.; Debon, C.; Hariton, C. J. Pharmacol. Toxicol. Methods 1996, 36, 223-228.

In one study, optical coherence tomography (OCT) confirmed the morphology of clear corneal incision wounds was not constant but varied in response to changes in the intraocular pressure. See McDonnell, P. J.; Taban, M.; Sarayba, M.; Rao, B.; Zhang, J.; Schiffman, R.; Chen, Z. P. Ophthalmology 2003, 110, 2342-2348. When the eyes were well pressurized (20 mm Hg or higher), the chambers were deeply formed, and the wound edges were well apposed. Elevation of intraocular pressure up to 40 to 50 mm Hg did not result in any separation of the wound edges. As the intraocular pressure was reduced to 10 mm Hg and below, the wound edges progressively separated. The separation began at the internal aspect of the wound, with posterior migration of the posterior and peripheral wound leaflet. This separation resulted in a wedge-shaped gaping in the internal aspect of the incision. Coincident with this wound margin separation, the spontaneous flow of aqueous humor through the wound was observed, and the chamber became shallower. Elevating the intraocular pressure resulted in prompt closure of the corneal wound at its superficial margin, termination of fluid leakage from the wound, and deepening of the anterior chamber. India ink was also applied to the surface of the cornea and quickly became visible through the operating microscope within the clear corneal incisions. Histologic examination of the wounds confirmed partial penetration of India ink particles along the edges of the incisions in every cornea. These studies demonstrated that a transient reduction of intraocular pressure might result in poor wound apposition in clear corneal incisions, with the potential for fluid flow across the cornea and into the anterior chamber, with the attendant risk of endophthalmitis. See McDonnell, P. J.; Taban, M.; Sarayba, M.; Rao, B.; Zhang, J.; Schiffman, R.; Chen, Z. P. Ophthalmology 2003, 110, 2342-2348.

Nonetheless, a progressive increase in the percentage of surgeons preferring self-sealing clear corneal incisions over scleral tunnel incisions in the United States and Europe has occurred over the past decade. See Leaming, D. V. J. Cataract Refract. Surg. 1995, 21, 378-385 and Leaming, D. V. J. Cataract Refract. Surg. 2001, 27, 948-955. Some studies, however, reveal an increased incidence of postoperative endophthalmitis after clear corneal cataract incisions and a recent, retrospective, case-controlled study, reported that clear corneal incisions were a statistically significant risk factor for acute post-cataract surgery endophthalmitis when compared with scleral tunnel incisions. See John, M. E.; Noblitt, R. Endophthalmitis. Scleral tunnel vs. clear corneal incision; Slack, Inc.: Thorofare, N J, 2001; Colleaux, K. M.; Hamilton, W. K. Can. J. Ophthalmol. 2000, 35, 373-378; Nagaki, Y.; Hayasaka, S.; Kadoi, C.; Matsumoto, M.; Yanagisawa, S.; Watanabe, K.; Watanabe, K.; Hayasaka, Y.; Ikeda, N.; Sato, S.; Kataoka, Y.; Togashi, M.; Abe, T. J. Cataract. Refract. Surg. 2003, 29, 20-26; Stonecipher, K. G.; Parmley, V. C.; Jensen, H.; Rowsey, J. J. Arch. Ophthalmol. 1991, 109, 1562-1563; Lertsumitkul, S.; Myers, P. C.; O'Rourke, M. T.; Chandra, J. Clin. Exp. Ophthalmol. 2001, 29, 400-405; and Blake, A. C.; Holekamp, N. M.; Bohigian, G.; Thompson, P. A. Am. J. Ophthalmol. 2003, 136, 300-305. The visual outcome following severe endophthalmitis is always guarded. In a Western Australian Endophthalmitis Study more than half of the subjects suffered visual impairment, with 41% poorer than 20/200, 53% poorer than 20/125, and 58% poorer than 20/40. See Semmens, J. B.; Li, J.; Morlet, N.; Ng, J. Clin. Exp. Ophthalmol. 2003, 31, 213-219. Post-cataract endophthalmitis remains a potentially blinding complication of a sight-restoring procedure. Refractive Surgery—Laser-assisted in situ Keratomileusis (LASIK)

Laser-assisted in situ keratomileusis is the popular refractive surgical procedure where a thin, hinged corneal flap is created by a microkeratome blade. This flap is then moved aside to allow an excimer laser beam to ablate the corneal stromal tissue with extreme precision for the correction of myopia (near-sightedness) and astigmatism. At the conclusion of the procedure, the flap is repositioned and allowed to heal. However, with trauma, this flap can become dislocated prior to healing, resulting in flap striae (folds) and severe visual loss. When this complication occurs, treatment involves prompt replacement of the flap and flap suturing. The use of sutures has limitations and drawbacks as discussed above. These novel adhesives could also play a useful role in the treatment of LASIK flap dislocations and striae (folds). These visually debilitating flap complications are seen not uncommonly following the popular procedure LASIK, and are currently treated by flap repositioning and suturing (which require considerable operative time and technical skill). A tissue adhesive could provide a more effective means to secure the flap.

### Refractive Surgery—Lens Replacement

Cataracts or other diseases or injuries that lead to poorly functioning or damaged lens require the natural lens to be replaced. The optical properties of the normal eye lens are the consequence of a high concentration of proteins called “crystallins” forming a natural hydrogel. In vertebrate lenses, a range of differently sized protein assemblies, the alpha-, beta- and gamma-crystallins, are found creating a medium of high refractive index. The anatomical basis of accommodation includes the lens substance, lens capsule, zonular fibers, ciliary muscle and the elastic part of the choroid. Accommodation occurs through accurately controlled adjustments in the shape and thickness of the lens. The capsular bag is essential in transmitting the various extralenticular forces to the lens substance.

Modern cataract surgery can be done through a small incision (usually 2.5-3.5 mm). Once the incision is made, the anterior chamber is filled with a viscoelastic and the capsular bag is pricked with a needle. From this incision, a small continuous circular capsulorhexis (CCC) approximately 1.5 mm in diameter is performed using capsulorhexis forceps. Next endocapsular phacoemulsification is performed and the lens epithelial cells are removed by aspiration.

The normal function of the lens is to focus light onto the retina. Since removing the cataract leaves the eye without a lens to focus light, an artificial (intraocular) lens is commonly placed inside the eye. Most intraocular lenses are made of plastic, silicone, or acrylic compounds; have no moving parts; and last for the remainder of a person's life. These intraocular lens implants are held in place by the posterior capsule are not able to provide ocular accommodation. Refilling the lens capsule with in situ crosslinking materials described herein offers the potential to produce a synthetic hydrogel with mechanical properties similar to the lens of a twenty year old. As such, the invention describes materials that reproduce the properties of the natural lens and these synthetic hydrogels maintain the integrity of the capsule to gain partial or full accommodation and restore vision to the patient.

### Retina—Retinal Holes

Techniques commonly used for the treatment of retinal holes, such as cryotherapy, diathermy and photocoagulation, are unsuccessful in the case of complicated retinal detachment, mainly because of the delay in the application and the weak strength of the chorioretinal adhesion. Cyanoacrylate retinopexy has been used in special cases. It has also been demonstrated that the chorioretinal adhesion is stronger and lasts longer than the earlier techniques. As noted previously with regard to corneal perforation treatment, the extremely rapid polymerization of cyanoacrylate glues (for example, risk of adhesion of the injector to the retina), the difficulty to use them in aqueous conditions and the toxicity are inconveniences and risks associated with this method. The polymerization can be slowed down by adding iophendylate to the monomers but still the reaction occurs in two to three seconds. Risks of retinal tear at the edge of the treated hole can also be observed because of the hardness of cyanoacrylate once polymerized.

### Retina—Vitrectomy/Sclerotomy Incisions

The vitreous is a normally clear, gel-like substance that fills the center of the eye. It makes up approximately ⅔ of the eye's volume, giving it form and shape before birth. Certain problems affecting the back of the eye may require a vitrectomy, or surgical removal of the vitreous. During a vitrectomy, the surgeon creates small incisions/punctures in the eye (sclerotomies) for separate instruments. These incisions are placed in the pars plana of the eye, which is located just behind the iris but in front of the retina. The instruments which pass through these incisions include a light pipe, an infusion port, and the vitrectomy cutting device. Upon completion of pars plana vitrectomy, each sclerotomy site is closed with a single interrupted suture of 8-0 silk or 7-0 polyglycolic acid suture. After a vitrectomy, the eye is filled with fluid until the vitreous is replaced as the eye secretes aqueous and nutritive fluids.

Some of the most common eye conditions that require vitrectomy include: 1) complications from diabetic retinopathy, such as retinal detachment or bleeding, 2) macular hole, 3) retinal detachment, 4) pre-retinal membrane fibrosis, 5) bleeding inside the eye (vitreous hemorrhage), 6) injury or infection, and 7) certain problems related to previous eye surgery.

### Glaucoma—Filtering Bleb

Leaking filtering blebs after glaucoma surgery are difficult to manage and can lead to serious, vision-threatening complications. Filtering blebs can result in hypotony and shallowing of the anterior chamber, choroidal effusion, maculopathy, retinal, and choroidal folds, suprachoroidal hemorrhage, corneal decompensation, peripheral anterior synechiae, and cataract formation. A filtering bleb can also lead to the loss of bleb function and to the severe complications of endophthalmaitis. The incidence of bleb leaks increases with the use of antimetabolites. Bleb leaks in eyes treated with 5-fluorouracil or mitomycin C may occur in as many as 20 to 40% of patients. Bleb leaks in eyes treated with antimetabolities may be difficult to heal because of thin avascular tissue and because of abnormal fibrovascular response. If the leak persists despite the use of conservative management, a 9-0 to 10-0 nylon or absorbable suture on a tapered vascular needle can be used to close the conjunctival wound. In a thin-walled or avascular bleb, a suture may not be advisable because it could tear the tissue and cause a larger leak. Fibrin adhesives have been used to close bleb leaks. The adhesive is applied to conjunctival wound simultaneously with thrombin to form a fibrin clot at the application site. The operative field must be dry during the application because fibrin will not adhere to wet tissue. Cyanoacrylate glue may be used to close a conjuctival opening. To apply the glue, the surrounding tissue must be dried and a single drop of the cyanoacrylate is placed. The operative surgeon must be careful not to seal the applicator to the tissue or to seal surrounding tissue with glue given its quick reaction. A soft contact lens is then applied over the glue to decrease patient discomfort. However, this procedure can actually worsen the problem if the cyanoacrylate tears from the bleb and causes a larger wound.

### Oculoplastics—Blepharoplasty Incisions

Blepharoplasty is an operation to remove excess skin and fat, and to reinforce surrounding muscle and tendons, around the eyes to correct droopy eyelids and bagginess under the eyes. It can be performed on the upper lids and lower lids, at the same time or separately. The operation may be done using either conventional or laser techniques. For surgery on the upper eyelids, cuts are made into the natural lines and creases in the lid, and into the laughter lines at the corner of the eye. For surgery on the lower eyelids, a cut is usually made just below the eyelashes. This means the scars run along the eye's natural folds, concealing them as much as possible. Excess fat and loose skin are removed, and the cut is closed using sutures. If only fat is being removed, sometimes the cut is made on the inside of the lower eyelid, leaving no visible scar. A tissue adhesive could provide a more effective means to secure the cuts made during surgery.

### Gastrointestinal Anastomosis

The sealants and methods of the present invention should be useful in gastrointestinal anastomosis procedures. Gastrointestinal anastomosis is the technique of joining two pieces of bowel together. There are many techniques for gastro-intestinal anastomosis, including both mechanical stapled techniques and hand-sutured procedures. The technique may involve a simple end-end anastomosis of two pieces of jejunum, a more complex colo-anal anastomosis, or a biliary enteric join. One problem with techniques employing sutures or staples is that leakage may occur around the sutures or staples. See, for example, Bruce et al. Br. J. Surg. 88:1157-1168 (2001) reporting leakage rates of 5-8%. However, sealants and methods of the invention could be used to supplement the sutures or staples used in intestinal anastomoses, providing a better seal that reduces leakage. Compositions and procedures for proper sealing the consequences of a failed anastomosis are severe and frequently life-threatening. Although failures can be caused by myriad factors, including poor surgical technique (e.g., sutures that were not inserted correctly; knots that were tied too tightly rendering the ends ischaemic; or incorrect use of a staple gun), the sealants and methods of the invention should decrease or eliminate some of the causes of failed gastrointestinal anastomosis procedures.

### Prostatectomy Urethral-Bladder Anastomosis

The sealants and methods of the present invention should be useful in prostatectomy urethral-bladder anastomosis procedures. Prostatectomy urethral-bladder anastomosis is the technique of joining together a patient's ureter and bladder after surgical removal of his prostate gland. Failures are caused by myriad factors, including poor surgical technique (e.g., sutures that were not inserted correctly; knots that were tied too tightly rendering the ends ischaemic). The sealants and methods of the invention should decrease or eliminate some of the causes of failed prostatectomy urethral-bladder anastomosis procedures.

### Cartilage, Meniscus and Disk Repair

Cartilaginous tissues play important roles in contributing to load support and energy dissipation in the joints of the musculoskeletal system. These tissues include articular cartilage which is predominantly an avascular and alymphatic tissue with very low cell-density. As a result, articular cartilage has limited capacity for self-repair following injury or aging. Degeneration of cartilage in the meniscus, interverebral disks, or joints can lead to severe and debilitating pain in patients. Injuries to these tissues are often retained for many years and may eventually lead to more severe secondary damage. See Moskowitz, R. W., Osteoarthritis: diagnosis and medical/surgical management. 2^{nd} ed.; W.B. Saunders Company: 1984. Today, more than one million knee, hip, and shoulder joint surgical procedures are performed annually in the United States as a consequence of trauma or a lifetime of wear and tear. See Praemer, A.; Furner, S.; Rice, D. P. Musculoskeletal Conditions in the United States, American Academy of Orthopaedic Surgeons: Rosemont, Ill., 1999. Despite the large number of patients suffering from cartilage degeneration, the only widely-available treatment options for cartilage degeneration are chronic administration of anti-inflammatory agents, total joint replacement, osteotomy, or allograft transplantation, each of which leads to mixed long-term results. The compositions and methods of the present invention should be useful in the treatment of such disorders and injuries.

### Tissue Plane Applications

The materials of the invention can be applied to two planes of tissue and then these two tissues can be sealed together. Over time the sealant/hydrogel degrades as new tissue grows into the area. Applications include a number of cosmetic and tissue restoration surgeries. The sealant is used when the procedures involve significant tissue plane separation that may result in formation of seroma with associated complications, such as infection, e.g., general surgery procedures, such as mastectomies and lumpectomies, and plastic surgery procedures, such as abdominoplastys, rhytidectomy or rhinoplastys, mammaplasty and other cosmetic or reconstructive surguries or procedures, forehead lifts and buttocks lifts, as well as skin grafts, biopsy closure, cleft-palate reconstruction, hernia repair, lymph node resection, groin repair, Caesarean section, laparoscopic trocar repair, vaginal tear repair, and hand surgery.

### Vascular and Cardiovascular Repair

The compositions and methods of the invention may be used for repairing, closing, and/or securing vascular and cardiovascular tissue. Representative procedures include coronary artery bypass grafts, coronary angioplasty, diagnostic cardiac catheterization, carotid endarterectomy, and valve repair. An additional use of the sealant is for the repair of cardiac tissue after a myocardial infarction. The polymer would be applied to the infarcted tissue to provide structural support to the weakened tissue. For example, the material would act as a sleeve for the cardiac tissue.

### Repair of Dura Tissue

Dura tissue is a fibrous membrane covering the brain and the spinal cord and lining the inner surface of the skull. Standard methods of dural repair involve the application of interrupted sutures and the use of dural replacement materials (duraplasty). This is a meticulous surgery and suffers from the limitation that pinholes produced by surgical needles can cause leakage. Moreover, intraoperative dehydration can shrink the dura creating a difficult closure since it is difficult to approximate the edges with sutures. In older patients, the dura is often more susceptiable to tearing when stretched and/or sutured because the dura can be thin and fragile. Adhesives such as fibrin have been explored for repair of dura tissue, but have had limited success. See “Glue in the Repair of Dural Defects in Craniofacial Resections,” J. Latyngology and Otology 1992, 106, 356-57; Kjaergard et al., “Autologous Fibrin Glue Preparation and Clinical Use in Thoracic Surgery,” Eur. J. Cardio-Thorc. Surg. 1992, 6, 52-54; Thompson et al., “Fibrin Glue: A Review of Its Preparation, Efficacy, and Adverse Effects as a Topical Hemostat,” Drug Intelligence and Clinical Pharmacy 1988, 22, 946-52; and Brennan, “Fibrin Glue,” Blood Reviews 1991, 5, 240-44. The sealants and methods of the present invention should be useful in repairing the dura after a craniotomy or laminectomy and prevent postoperative leakage of cerebrospinal fluid. See Preul et al. Neurosurgery 2003, 53, 1189-1199 and Balance, C. A. in “Some Points in the Surgery of the Brain and Its Membranes” London, Macmillan & Co.

### Injection Site Wound

Many therapeutic agents are administered to a patient by injection. However, one complication of this procedure is that the tissue at the injection site can become infected or susceptible to poor healing. One clinical situation where infections are prone to occur is when a therapeutic agent is injected into the eye of a patient. This mode of administration is used in the treatment of age-related macular degeneration (AMD) and results in about 2% of patients suffering from infection or endophthalmitis.

Age-related macular degeneration is a disease that blurs the sharp, central vision needed for “straight-ahead” activities such as reading and driving. Specifically, AMD is a progressive disease of the retina where the light-sensing cells in the central area of vision (the macula) stop working and eventually die. The disease is caused by a combination of genetic and environmental factors, and it is most common in people who are age sixty and over. In fact, AMD is the leading cause of visual impairment in the elderly population. It is estimated that fifteen million people in the United States have AMD, with approximately two million new cases diagnosed annually. There are two types of AMD—wet and dry. Wet AMD occurs when abnormal blood vessels behind the retina start to grow under the macula. These new blood vessels tend to be very fragile and often leak blood and fluid. The blood and fluid raise the macula from its normal place at the back of the eye. Damage to the macula occurs rapidly and loss of central vision can occur quickly. On the other hand, dry AMD occurs when the light-sensitive cells in the macula slowly break down, gradually blurring central vision in the affected eye. Central vision is gradually lost. In this disease, Vascular Endothelial Growth Factor (VEGF) is a key growth factor, which promotes the new growth blood vessels. Currently, it is believed that that when the retinal pigment epithelial (RPE) cells begin to wither from lack of nutrition (i.e., ischemia), VEGF is up-regulated and new vessels are created. Yet, the vessels do not form properly and leaking results. This leakage causes scarring in the macula and eventual loss of central vision. To prevent or inhibit this neovascularization process, antiangiogenic drugs are given the patient. In most cases, the drugs are injected into the vitreous of the eyeball, then pass into the subretinal space where the vessels proliferate. These drugs include mucagenm squalamine lactate, combretastatin 4 prodrug, and avastin.

The sealants and methods of the present invention should be useful in sealing injection site wounds. Among the various possibilities, the injection can be given and then the sealant applied to the injection site, or alternatively the sealant can be applied and then the injection can be done through the sealant.

### Biologically Active Agents and Pharmaceutically Active Agents

In certain embodiments, biologically active agents may be incorporated in the biocompatible adhesive materials of the invention. Active agents amenable for use in the compositions of the present invention include growth factors, such as transforming growth factors (TGFs), fibroblast growth factors (FGFs), platelet derived growth factors (PDGFs), epidermal growth factors (EGFs), connective tissue activated peptides (CTAPs), osteogenic factors, and biologically active analogs, fragments, and derivatives of such growth factors. Members of the transforming growth factor (TGF) supergene family, which are multifunctional regulatory proteins, are particularly preferred. Members of the TGF supergene family include the beta transforming growth factors (for example, TGF-β1, TGF-β2, TGF-β3); bone morphogenetic proteins (for example, BMP-1, BMP-2, BMP-3, BMP-4, BMP-5, BMP-6, BMP-7, BMP-8, BMP-9); heparin-binding growth factors (for example, fibroblast growth factor (FGF), epidermal growth factor (EGF), platelet-derived growth factor (PDGF), insulin-like growth factor (IGF)); Inhibins (for example, Inhibin A, Inhibin B); growth differentiating factors (for example, GDF-1); and Activins (for example, Activin A, Activin B, Activin AB).

In addition to the biological active agents discussed above, a large number of pharmaceutical agents are known in the art and are amenable for use in the biocompatible adhesive materials of the invention. The term “pharmaceutical agent” includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

Non-limiting examples of broad categories of useful pharmaceutical agents include the following therapeutic categories: anabolic agents, antacids, anti-asthmatic agents, anti-cholesterolemic and anti-lipid agents, anti-coagulants, anti-convulsants, antidiarrheals, anti-emetics, anti-infective agents, anti-inflammatory agents, anti-manic agents, anti-nauseants, anti-neoplastic agents, anti-obesity agents, anti-pyretic and analgesic agents, anti-spasmodic agents, anti-thrombotic agents, anti-uricemic agents, anti-anginal agents, antihistamines, anti-tussives, appetite suppressants, biologicals, cerebral dilators, coronary dilators, decongestants, diuretics, diagnostic agents, erythropoietic agents, expectorants, gastrointestinal sedatives, hyperglycemic agents, hypnotics, hypoglycemic agents, ion exchange resins, laxatives, mineral supplements, mucolytic agents, neuromuscular drugs, peripheral vasodilators, psychotropics, sedatives, stimulants, thyroid and anti-thyroid agents, uterine relaxants, vitamins, and prodrugs.

More specifically, non-limiting examples of useful pharmaceutical agents include the following therapeutic categories: analgesics, such as nonsteroidal anti-inflammatory drugs, opiate agonists and salicylates; antihistamines, such as H₁-blockers and H₂-blockers; anti-infective agents, such as anthelmintics, antianaerobics, antibiotics, aminoglycoside antibiotics, antifungal antibiotics, cephalosporin antibiotics, macrolide antibiotics, miscellaneous beta-lactam antibiotics, penicillin antibiotics, quinolone antibiotics, sulfonamide antibiotics, tetracycline antibiotics, antimycobacterials, antituberculosis antimycobacterials, antiprotozoals, antimalarial antiprotozoals, antiviral agents, anti-retroviral agents, scabicides, and urinary anti-infectives; antineoplastic agents, such as alkylating agents, nitrogen mustard aklylating agents, nitrosourea alkylating agents, antimetabolites, purine analog antimetabolites, pyrimidine analog antimetabolites, hormonal antineoplastics, natural antineoplastics, antibiotic natural antineoplastics, and vinca alkaloid natural antineoplastics; autonomic agents, such as anticholinergics, antimuscarinic anticholinergics, ergot alkaloids, parasympathomimetics, cholinergic agonist parasympathomimetics, cholinesterase inhibitor para-sympathomimetics, sympatholytics, alpha-blocker sympatholytics, beta-blocker sympatholytics, sympathomimetics, and adrenergic agonist sympathomimetics; cardiovascular agents, such as antianginals, beta-blocker antianginals, calcium-channel blocker antianginals, nitrate antianginals, antiarrhythmics, cardiac glycoside antiarrhythmics, class I antiarrhythmics, class II antiarrhythmics, class III antiarrhythmics, class IV antiarrhythmics, antihypertensive agents, alpha-blocker antihypertensives, angiotensin-converting enzyme inhibitor (ACE inhibitor) antihypertensives, beta-blocker antihypertensives, calcium-channel blocker antihypertensives, central-acting adrenergic antihypertensives, diuretic antihypertensive agents, peripheral vasodilator antihypertensives, antilipemics, bile acid sequestrant antilipemics, HMG-CoA reductase inhibitor antilipemics, inotropes, cardiac glycoside inotropes, and thrombolytic agents; dermatological agents, such as antihistamines, anti-inflammatory agents, corticosteroid anti-inflammatory agents, antipruritics/local anesthetics, topical anti-infectives, antifungal topical anti-infectives, antiviral topical anti-infectives, and topical antineoplastics; electrolytic and renal agents, such as acidifying agents, alkalinizing agents, diuretics, carbonic anhydrase inhibitor diuretics, loop diuretics, osmotic diuretics, potassium-sparing diuretics, thiazide diuretics, electrolyte replacements, and uricosuric agents; enzymes, such as pancreatic enzymes and thrombolytic enzymes; gastrointestinal agents, such as antidiarrheals, antiemetics, gastrointestinal anti-inflammatory agents, salicylate gastrointestinal anti-inflammatory agents, antacid anti-ulcer agents, gastric acid-pump inhibitor anti-ulcer agents, gastric mucosal anti-ulcer agents, H₂-blocker anti-ulcer agents, cholelitholytic agents, digestants, emetics, laxatives and stool softeners, and prokinetic agents; general anesthetics, such as inhalation anesthetics, halogenated inhalation anesthetics, intravenous anesthetics, barbiturate intravenous anesthetics, benzodiazepine intravenous anesthetics, and opiate agonist intravenous anesthetics; hematological agents, such as antianemia agents, hematopoietic antianemia agents, coagulation agents, anticoagulants, hemostatic coagulation agents, platelet inhibitor coagulation agents, thrombolytic enzyme coagulation agents, and plasma volume expanders; hormones and hormone modifiers, such as abortifacients, adrenal agents, corticosteroid adrenal agents, androgens, anti-androgens, antidiabetic agents, sulfonylurea antidiabetic agents, antihypoglycemic agents, oral contraceptives, progestin contraceptives, estrogens, fertility agents, oxytocics, parathyroid agents, pituitary hormones, progestins, antithyroid agents, thyroid hormones, and tocolytics; immunobiologic agents, such as immunoglobulins, immunosuppressives, toxoids, and vaccines; local anesthetics, such as amide local anesthetics and ester local anesthetics; musculoskeletal agents, such as anti-gout anti-inflammatory agents, corticosteroid anti-inflammatory agents, gold compound anti-inflammatory agents, immuno-suppressive anti-inflammatory agents, nonsteroidal anti-inflammatory drugs (NSAIDs), salicylate anti-inflammatory agents, skeletal muscle relaxants, neuromuscular blocker skeletal muscle relaxants, and reverse neuromuscular blocker skeletal muscle relaxants; neurological agents, such as anticonvulsants, barbiturate anticonvulsants, benzodiazepine anticonvulsants, anti-migraine agents, anti-parkinsonian agents, anti-vertigo agents, opiate agonists, and opiate antagonists; ophthalmic agents, such as anti-glaucoma agents, beta-blocker anti-gluacoma agents, miotic anti-glaucoma agents, mydriatics, adrenergic agonist mydriatics, antimuscarinic mydriatics, ophthalmic anesthetics, ophthalmic anti-infectives, ophthalmic aminoglycoside anti-infectives, ophthalmic macrolide anti-infectives, ophthalmic quinolone anti-infectives, ophthalmic sulfonamide anti-infectives, ophthalmic tetracycline anti-infectives, ophthalmic anti-inflammatory agents, ophthalmic corticosteroid anti-inflammatory agents, and ophthalmic nonsteroidal anti-inflammatory drugs (NSAIDs); psychotropic agents, such as antidepressants, heterocyclic antidepressants, monoamine oxidase inhibitors (MAOIs), selective serotonin re-uptake inhibitors (SSRIs), tricyclic antidepressants, antimanics, antipsychotics, phenothiazine antipsychotics, anxiolytics, sedatives, and hypnotics, barbiturate sedatives and hypnotics, benzodiazepine anxiolytics, sedatives, and hypnotics, and psychostimulants; respiratory agents, such as antitussives, bronchodilators, adrenergic agonist bronchodilators, antimuscarinic bronchodilators, expectorants, mucolytic agents, respiratory anti-inflammatory agents, and respiratory corticosteroid anti-inflammatory agents; toxicology agents, such as antidotes, heavy metal antagonists/chelating agents, substance abuse agents, deterrent substance abuse agents, and withdrawal substance abuse agents; minerals; and vitamins, such as vitamin A, vitamin B, vitamin C, vitamin D, vitamin E, and vitamin K.

Preferred classes of useful pharmaceutical agents from the above categories include: (1) nonsteroidal anti-inflammatory drugs (NSAIDs) analgesics, such as diclofenac, ibuprofen, ketoprofen, and naproxen; (2) opiate agonist analgesics, such as codeine, fentanyl, hydromorphone, and morphine; (3) salicylate analgesics, such as aspirin (ASA) (enteric coated ASA); (4) H₁-blocker antihistamines, such as clemastine and terfenadine; (5) H₂-blocker antihistamines, such as cimetidine, famotidine, nizadine, and ranitidine; (6) anti-infective agents, such as mupirocin; (7) antianaerobic anti-infectives, such as chloramphenicol and clindamycin; (8) antifungal antibiotic anti-infectives, such as amphotericin b, clotrimazole, fluconazole, and ketoconazole; (9) macrolide antibiotic anti-infectives, such as azithromycin and erythromycin; (10) miscellaneous beta-lactam antibiotic anti-infectives, such as aztreonam and imipenem; (11) penicillin antibiotic anti-infectives, such as nafcillin, oxacillin, penicillin G, and penicillin V; (12) quinolone antibiotic anti-infectives, such as ciprofloxacin and norfloxacin; (13) tetracycline antibiotic anti-infectives, such as doxycycline, minocycline, and tetracycline; (14) antituberculosis antimycobacterial anti-infectives such as isoniazid (INH), and rifampin; (15) antiprotozoal anti-infectives, such as atovaquone and dapsone; (16) antimalarial antiprotozoal anti-infectives, such as chloroquine and pyrimethamine; (17) anti-retroviral anti-infectives, such as ritonavir and zidovudine; (18) antiviral anti-infective agents, such as acyclovir, ganciclovir, interferon alfa, and rimantadine; (19) alkylating antineoplastic agents, such as carboplatin and cisplatin; (20) nitrosourea alkylating antineoplastic agents, such as carmustine (BCNU); (21) antimetabolite antineoplastic agents, such as methotrexate; (22) pyrimidine analog antimetabolite antineoplastic agents, such as fluorouracil (5-FU) and gemcitabine; (23) hormonal antineoplastics, such as goserelin, leuprolide, and tamoxifen; (24) natural antineoplastics, such as aldesleukin, interleukin-2, docetaxel, etoposide (VP-16), interferon alfa, paclitaxel, and tretinoin (ATRA); (25) antibiotic natural antineoplastics, such as bleomycin, dactinomycin, daunorubicin, doxorubicin, and mitomycin; (26) vinca alkaloid natural antineoplastics, such as vinblastine and vincristine; (27) autonomic agents, such as nicotine; (28) anticholinergic autonomic agents, such as benztropine and trihexyphenidyl; (29) antimuscarinic anticholinergic autonomic agents, such as atropine and oxybutynin; (30) ergot alkaloid autonomic agents, such as bromocriptine; (31) cholinergic agonist parasympathomimetics, such as pilocarpine; (32) cholinesterase inhibitor parasympathomimetics, such as pyridostigmine; (33) alpha-blocker sympatholytics, such as prazosin; (34) beta-blocker sympatholytics, such as atenolol; (35) adrenergic agonist sympathomimetics, such as albuterol and dobutamine; (36) cardiovascular agents, such as aspirin (ASA) (enteric coated ASA); (37) beta-blocker antianginals, such as atenolol and propranolol; (38) calcium-channel blocker antianginals, such as nifedipine and verapamil; (39) nitrate antianginals, such as isosorbide dinitrate (ISDN); (40) cardiac glycoside antiarrhythmics, such as digoxin; (41) class I anti-arrhythmics, such as lidocaine, mexiletine, phenytoin, procainamide, and quinidine; (42) class II antiarrhythmics, such as atenolol, metoprolol, propranolol, and timolol; (43) class III antiarrhythmics, such as amiodarone; (44) class IV antiarrhythmics, such as diltiazem and verapamil; (45) α-blocker antihypertensives, such as prazosin; (46) angiotensin-converting enzyme inhibitor (ACE inhibitor) antihypertensives, such as captopril and enalapril; (47) β-blocker antihypertensives, such as atenolol, metoprolol, nadolol, and propanolol; (48) calcium-channel blocker antihypertensive agents, such as diltiazem and nifedipine; (49) central-acting adrenergic antihypertensives, such as clonidine and methyldopa; (50) diurectic antihypertensive agents, such as amiloride, furosemide, hydrochlorothiazide (HCTZ), and spironolactone; (51) peripheral vasodilator antihypertensives, such as hydralazine and minoxidil; (52) antilipemics, such as gemfibrozil and probucol; (53) bile acid sequestrant antilipemics, such as cholestyramine; (54) HMG-CoA reductase inhibitor antilipemics, such as lovastatin and pravastatin; (55) inotropes, such as amrinone, dobutamine, and dopamine; (56) cardiac glycoside inotropes, such as digoxin; (57) thrombolytic agents, such as alteplase (TPA), anistreplase, streptokinase, and urokinase; (58) dermatological agents, such as colchicine, isotretinoin, methotrexate, minoxidil, tretinoin (ATRA); (59) dermatological corticosteroid anti-inflammatory agents, such as betamethasone and dexamethasone; (60) antifungal topical anti-infectives, such as amphotericin B, clotrimazole, miconazole, and nystatin; (61) antiviral topical anti-infectives, such as acyclovir; (62) topical antineoplastics, such as fluorouracil (5-FU); (63) electrolytic and renal agents, such as lactulose; (64) loop diuretics, such as furosemide; (65) potassium-sparing diuretics, such as triamterene; (66) thiazide diuretics, such as hydro-chlorothiazide (HCTZ); (67) uricosuric agents, such as probenecid; (68) enzymes such as RNase and DNase; (69) thrombolytic enzymes, such as alteplase, anistreplase, streptokinase and urokinase; (70) antiemetics, such as prochlorperazine; (71) salicylate gastrointestinal anti-inflammatory agents, such as sulfasalazine; (72) gastric acid-pump inhibitor anti-ulcer agents, such as omeprazole; (73) H₂-blocker anti-ulcer agents, such as cimetidine, famotidine, nizatidine, and ranitidine; (74) digestants, such as pancrelipase; (75) prokinetic agents, such as erythromycin; (76) opiate agonist intravenous anesthetics such as fentanyl; (77) hematopoietic antianemia agents, such as erythropoietin, filgrastim (G-CSF), and sargramostim (GM-CSF); (78) coagulation agents, such as antihemophilic factors 1-10 (AHF 1-10); (79) anticoagulants, such as warfarin; (80) thrombolytic enzyme coagulation agents, such as alteplase, anistreplase, streptokinase and urokinase; (81) hormones and hormone modifiers, such as bromocriptine; (82) abortifacients, such as methotrexate; (83) antidiabetic agents, such as insulin; (84) oral contraceptives, such as estrogen and progestin; (85) progestin contraceptives, such as levonorgestrel and norgestrel; (86) estrogens such as conjugated estrogens, diethylstilbestrol (DES), estrogen (estradiol, estrone, and estropipate); (87) fertility agents, such as clomiphene, human chorionic gonadatropin (HCG), and menotropins; (88) parathyroid agents such as calcitonin; (89) pituitary hormones, such as desmopressin, goserelin, oxytocin, and vasopressin (ADH); (90) progestins, such as medroxyprogesterone, norethindrone, and progesterone; (91) thyroid hormones, such as levothyroxine; (92) immunobiologic agents, such as interferon beta-1b and interferon gamma-1b; (93) immunoglobulins, such as immune globulin IM, IMIG, IGIM and immune globulin IV, IVIG, IGIV; (94) amide local anesthetics, such as lidocaine; (95) ester local anesthetics, such as benzocaine and procaine; (96) musculoskeletal corticosteroid anti-inflammatory agents, such as beclomethasone, betamethasone, cortisone, dexamethasone, hydrocortisone, and prednisone; (97) musculoskeletal anti-inflammatory immunosuppressives, such as azathioprine, cyclophosphamide, and methotrexate; (98) musculoskeletal nonsteroidal anti-inflammatory drugs (NSAIDs), such as diclofenac, ibuprofen, ketoprofen, ketorlac, and naproxen; (99) skeletal muscle relaxants, such as baclofen, cyclobenzaprine, and diazepam; (100) reverse neuromuscular blocker skeletal muscle relaxants, such as pyridostigmine; (101) neurological agents, such as nimodipine, riluzole, tacrine and ticlopidine; (102) anticonvulsants, such as carbamazepine, gabapentin, lamotrigine, phenytoin, and valproic acid; (103) barbiturate anticonvulsants, such as phenobarbital and primidone; (104) benzodiazepine anticonvulsants, such as clonazepam, diazepam, and lorazepam; (105) anti-parkisonian agents, such as bromocriptine, levodopa, carbidopa, and pergolide; (106) anti-vertigo agents, such as meclizine; (107) opiate agonists, such as codeine, fentanyl, hydromorphone, methadone, and morphine; (108) opiate antagonists, such as naloxone; (109) β-blocker anti-glaucoma agents, such as timolol; (110) miotic anti-glaucoma agents, such as pilocarpine; (111) ophthalmic aminoglycoside antiinfectives, such as gentamicin, neomycin, and tobramycin; (112) ophthalmic quinolone anti-infectives, such as ciprofloxacin, norfloxacin, and ofloxacin; (113) ophthalmic corticosteroid anti-inflammatory agents, such as dexamethasone and prednisolone; (114) ophthalmic nonsteroidal anti-inflammatory drugs (NSAIDs), such as diclofenac; (115) antipsychotics, such as clozapine, haloperidol, and risperidone; (116) benzodiazepine anxiolytics, sedatives and hypnotics, such as clonazepam, diazepam, lorazepam, oxazepam, and prazepam; (117) psychostimulants, such as methylphenidate and pemoline; (118) antitussives, such as codeine; (119) bronchodilators, such as theophylline; (120) adrenergic agonist bronchodilators, such as albuterol; (121) respiratory corticosteroid anti-inflammatory agents, such as dexamethasone; (122) antidotes, such as flumazenil and naloxone; (123) heavy metal antagonists/chelating agents, such as penicillamine; (124) deterrent substance abuse agents, such as disulfiram, naltrexone, and nicotine; (125) withdrawal substance abuse agents, such as bromocriptine; (126) minerals, such as iron, calcium, and magnesium; (127) vitamin B compounds, such as cyanocobalamin (vitamin B12) and niacin (vitamin B3); (128) vitamin C compounds, such as ascorbic acid; and (129) vitamin D compounds, such as calcitriol.

In addition to the foregoing, the following less common drugs may also be used: chlorhexidine; estradiol cypionate in oil; estradiol valerate in oil; flurbiprofen; flurbiprofen sodium; ivermectin; levodopa; nafarelin; and somatropin. Further, the following drugs may also be used: recombinant beta-glucan; bovine immunoglobulin concentrate; bovine superoxide dismutase; the formulation comprising fluorouracil, epinephrine, and bovine collagen; recombinant hirudin (r-Hir), HIV-1 immunogen; human anti-TAC antibody; recombinant human growth hormone (r-hGH); recombinant human hemoglobin (r-Hb); recombinant human mecasermin (r-IGF-1); recombinant interferon β-1a; lenograstim (G-CSF); olanzapine; recombinant thyroid stimulating hormone (r-TSH); and topotecan.

Further still, the following intravenous products may be used: acyclovir sodium; aldesleukin; atenolol; bleomycin sulfate, human calcitonin; salmon calcitonin; carboplatin; carmustine; dactinomycin, daunorubicin HCl; docetaxel; doxorubicin HCl; epoetin alfa; etoposide (VP-16); fluorouracil (5-FU); ganciclovir sodium; gentamicin sulfate; interferon alfa; leuprolide acetate; meperidine HCl; methadone HCl; methotrexate sodium; paclitaxel; ranitidine HCl; vinblastin sulfate; and zidovudine (AZT).

Further specific examples of useful pharmaceutical agents from the above categories include: (a) anti-neoplastics such as androgen inhibitors, antimetabolites, cytotoxic agents, and immunomodulators; (b) anti-tussives such as dextromethorphan, dextromethorphan hydrobromide, noscapine, carbetapentane citrate, and chlorphedianol hydrochloride; (c) antihistamines such as chlorpheniramine maleate, phenindamine tartrate, pyrilamine maleate, doxylamine succinate, and phenyltoloxamine citrate; (d) decongestants such as phenylephrine hydrochloride, phenylpropanolamine hydrochloride, pseudoephedrine hydrochloride, and ephedrine; (e) various alkaloids such as codeine phosphate, codeine sulfate and morphine; (f) mineral supplements such as potassium chloride, zinc chloride, calcium carbonates, magnesium oxide, and other alkali metal and alkaline earth metal salts; (g) ion exchange resins such as cholestryramine; (h) anti-arrhythmics such as N-acetylprocainamide; (i) antipyretics and analgesics such as acetaminophen, aspirin and ibuprofen; (j) appetite suppressants such as phenyl-propanolamine hydrochloride or caffeine; (k) expectorants such as guaifenesin; (l) antacids such as aluminum hydroxide and magnesium hydroxide; (m) biologicals such as peptides, polypeptides, proteins and amino acids, hormones, interferons or cytokines, and other bioactive peptidic compounds, such as interleukins 1-18 including mutants and analogues, RNase, DNase, luteinizing hormone releasing hormone (LHRH) and analogues, gonadotropin releasing hormone (GnRH), transforming growth factor-.beta. (TGF-beta), fibroblast growth factor (FGF), tumor necrosis factor-alpha & beta (TNF-alpha & beta), nerve growth factor (NGF), growth hormone releasing factor (GHRF), epidermal growth factor (EGF), fibroblast growth factor homologous factor (FGFHF), hepatocyte growth factor (HGF), insulin growth factor (IGF), invasion inhibiting factor-2 (IIF-2), bone morphogenetic proteins 1-7 (BMP 1-7), somatostatin, thymosin-alpha-1, gamma-globulin, superoxide dismutase (SOD), complement factors, hGH, tPA, calcitonin, ANF, EPO and insulin; and (n) anti-infective agents such as antifungals, anti-virals, antiseptics and antibiotics.

Alternatively, the pharmaceutical agent may be a radiosensitizer, such as metoclopramide, sensamide or neusensamide (manufactured by Oxigene); profiromycin (made by Vion); RSR13 (made by Allos); Thymitaq (made by Agouron), etanidazole or lobenguane (manufactured by Nycomed); gadolinium texaphrin (made by Pharmacyclics); BuDR/Broxine (made by NeoPharm); IPdR (made by Sparta); CR2412 (made by Cell Therapeutic); L1X (made by Terrapin); or the like. Preferably, the biologically active substance is selected from the group consisting of peptides, poly-peptides, proteins, amino acids, polysaccharides, growth factors, hormones, anti-angiogenesis factors, interferons or cytokines, and pro-drugs. In a particularly preferred embodiment, the biologically active substance is a therapeutic drug or pro-drug, most preferably a drug selected from the group consisting of chemotherapeutic agents and other anti-neoplastics such as paclitaxel, antibiotics, anti-virals, antifungals, anti-inflammatories, and anticoagulants.

The biologically active substances are used in amounts that are therapeutically effective. While the effective amount of a biologically active substance will depend on the particular material being used, amounts of the biologically active substance from about 1% to about 65% may be desirable. Lesser amounts may be used to achieve efficacious levels of treatment for certain biologically active substances.

### Sterilization Procedures

A variety of procedures are known in the art for sterilizing a chemical composition. Sterilization may be accomplished by chemical, physical, or irradiation techniques. Examples of chemical methods include exposure to ethylene oxide or hydrogen peroxide vapor. Examples of physical methods include sterilization by heat (dry or moist), retort canning, and filtration. The British Pharmacopoeia recommends heating at a minimum of 160° C. for not less than 2 hours, a minimum of 170° C. for not less than 1 hour and a minimum of 180° C. for not less than 30 minutes for effective sterilization. For examples of heat sterilization, see U.S. Pat. No. 6,136,326, which is hereby incorporated by reference. Passing the chemical composition through a membrane can be used to sterilize a composition. For example, the composition is filtered through a small pore filter such as a 0.22 micron filter which comprises material inert to the composition being filtered. In certain embodiments, the filtration is conducted in a Class 100,000 or better clean room. Examples of irradiation methods include gamma irradiation, electron beam irradiation, microwave irradiation, and irradiation using visible light. One preferred method is electron beam irradiation, as described in U.S. Pat. Nos. 6,743,858; 6,248,800; and 6,143,805, each of which is hereby incorporated by reference.

There are several sources for electron beam irradiation. The two main groups of electron beam accelerators are: (1) a Dynamitron, which uses an insulated core transformer, and (2) radio frequency (RF) linear accelerators (linacs). The Dynamitron is a particle accelerator (4.5 MeV) designed to impart energy to electrons. The high energy electrons are generated and accelerated by the electrostatic fields of the accelerator electrodes arranged within the length of the glass-insulated beam tube (acceleration tube). These electrons, traveling through an extension of the evacuation beam tube and beam transport (drift pipe) are subjected to a magnet deflection system in order to produce a “scanned” beam, prior to leaving the vacuum enclosure through a beam window. The dose can be adjusted with the control of the percent scan, the beam current, and the conveyor speed. In certain embodiments, the electron-beam radiation employed may be maintained at an initial fluence of at least about 2 μCurie/cm², at least about 5 μCurie/cm², at least about 8 μCurie/cm², or at least about 10 μCurie/cm². In certain embodiments, the electron-beam radiation employed has an initial fluence of from about 2 to about 25 μCurie/cm². In certain embodiments, the electron-beam dosage is from about 5 to 50 kGray, or from about 15 to about 20 kGray with the specific dosage being selected relative to the density of material being subjected to electron-beam radiation as well as the amount of bioburden estimated to be therein. Such factors are well within the skill of the art.

The composition to be sterilized may be in any type of at least partially electron beam permeable container such as glass or plastic. In embodiments of the present invention, the container may be sealed or have an opening. Examples of glass containers include ampuoles, vials, syringes, pipettes, applicators, and the like. The penetration of electron beam irradiation is a function of the packaging. If there is not enough penetration from the side of a stationary electron beam, the container may be flipped or rotated to achieve adequate penetration. Alternatively, the electron beam source can be moved about a stationary package. In order to determine the dose distribution and dose penetration in product load, a dose map can be performed. This will identify the minimum and maximum dose zone within a product.

Procedures for sterilization using visible light are described in U.S. Pat. No. 6,579,916, which is hereby incorporated by reference. The visible light for sterilization can be generated using any conventional generator of sufficient power and breadth of wavelength to effect sterilization. Generators are commercially available under the tradename PureBright® in-line sterilization systems from PurePulse Technologies, Inc. 4241 Ponderosa Ave, San Diego, Calif. 92123, USA. The PureBright® in-line sterilization system employs visible light to sterilize clear liquids at an intensity approximately 90,000 times greater than surface sunlight. If the amount of UV light penetration is of concern, conventional UV absorbing materials can be used to filter out the UV light.

In certain embodiments, the composition is sterilized to provide a Sterility Assurance Level (SAL) of at least about 10⁻³. The Sterility Assurance Level measurement standard is described, for example, in ISO/CD 14937, the entire disclosure of which is incorporated herein by reference. In certain embodiments, the Sterility Assurance Level may be at least about 10⁻⁴, at least about 10⁻⁵, or at least about 10⁻⁶.

In certain embodiments of the present invention, one or more of the compositions, reagents, or components of a kit has been sterilized. The sterilization may be achieved using gamma radiation, e-beam radiation, dry heat sterilization, ethylene oxide sterilization, or a combination of any of them. The compositions, reagents, or components of the kits can be sterilized in an aqueous solution or neat.

In certain embodiments, the present invention relates to the aforementioned method, wherein said sterilizing is performed by treatment with ethylene oxide, hydrogen peroxide, heat, gamma irradiation, electron beam irradiation, microwave irradiation, or visible light irradiation.

### Delivery Systems

The adhesives of the present invention may be delivered, for example, to the wound, void, or damaged tissue of a patient using a large number of known delivery devices. For example, the delivery system may be a single-barrel syringe system. In certain embodiments, the single-barrel syringe is a double acting, single-barrel syringe system. In certain situations, a double- or multi-barrel syringe system may be preferable. In some instances, a delivery device that flows two or more streams of liquid in a mixing chamber may be preferable. Alternatively, a delivery device that mixes two solids and two liquids and then separately flows these streams of liquid to a mixing chamber may be advantageous. In certain embodiments, delivery may be assisted with machines, compressed air or gases, and the like. Of course, variations may be made in the size of the delivery device, the length of the delivery device, and/or the use of machines to aid in delivery.

### EXAMPLES

In order that the invention described herein may be more fully understood, the following examples are set forth. The examples described in this application are offered to illustrate the compounds, compositions, materials, device, and methods provided herein and are not to be construed in any way as limiting their scope.

### Example 1

### Burst Pressure Test

### Test Methods and Set up

The purpose of this procedure is to assess the maximum burst pressure or rupture strength of sealants on soft tissue. This test method provides a means for comparison of the performance of different sealants. This protocol is an adaptation of the Standard Test Method for Burst 10 Strength of Surgical Sealants; Designation F2392-04 reapproved in 2015 and can be used as a clinically relevant model for quality assurance, development, and comparative testing of different adhesives.

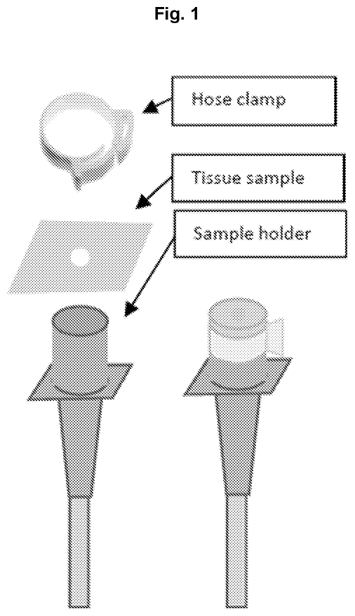

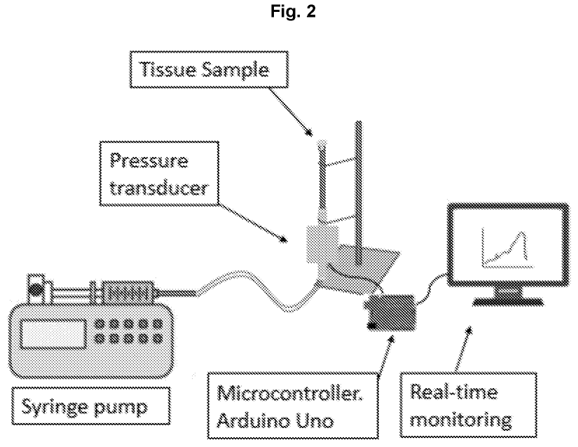

The testing machine (see FIGS. 1 and 2) for determining the sealant strength and system failure mechanism comprises essentially the following:
Test Fixture, a stationary fixture containing the test substrate and applied sealant. Fluid flows into the fixture at a fixed rate, allowing for the pressurization of the sealed substrate.
Syringe pump model NE-1000 Programmable Single Syringe Pump from Pump systems inc. is used in this protocol. Saline or 20 water is the testing fluid.
Pressure transducer or gage, PendoTech PREPS-N-000 PressureMAT bought from coleparmer is used to measure the pressure in the fluid line.
Data acquisition system, including a microcontroller and monitor 25 able to transduce the signal produced by the sensor to a recording computer

To perform burst pressure test, small porcine intestine is freshly harvested. Ideally, the

tissue is used within 24 h of harvest and should be kept between 5 and 10° C. The specimens are brought to room temperature prior to application of the sealant. To prepare the substrate to perform the burst test, a sample of tissue is cut in squares with 3.0 cm length for each side. With a hole puncher of 3 mm a hole is created in the center of the sample. When the aim is to
understand the performance over suture line, a 3 mm incision is made by a scalpel blade and closed with a single suture point.

After fixing the sample substrate on the holder, as shown in FIG. 1, 100 μL of sealant is homogeneously distributed to cover the hole/incision. After 5 minutes of sealant application burst test can be performed. Syringe pump is set to an infusion rate of 10 mL/min. The maximum burst pressure for each test is recorded and repeated at least 3 times due the large variability associated to differences in tissue substrate.

### Results

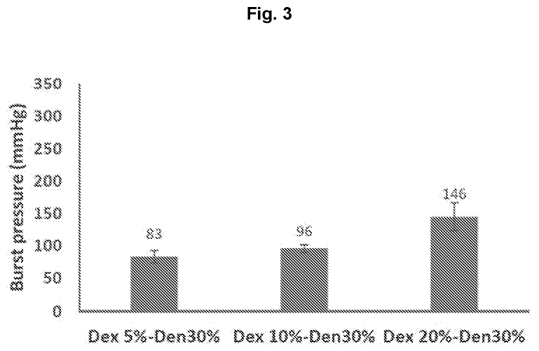

FIG. 3 depicts that an increase in dextran aldehyde solid content has a positive effect on the burst pressure, increasing the burst pressure values from 83 mmHg when dextran is fixed to 5% to 146 mmHg with 20% dextran.

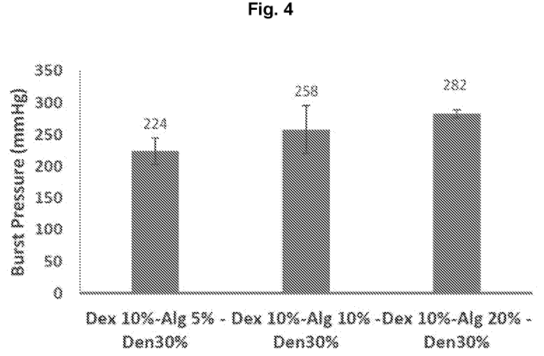

FIG. 4 depicts the impact of the addition of oxidized alginate to the dendrimer:dextran system. It can be appreciated that the addition of oxidized alginate (5, 10 or 20%) almost doubles the burst pressure values.

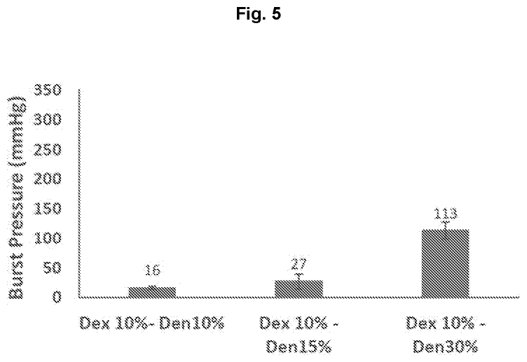

FIG. 5 depicts the high sensitivity of the burst pressure to dendrimer solid content. The increase in dendrimer solid content from 10 to 30 wt % increases the burst pressure almost ten times.

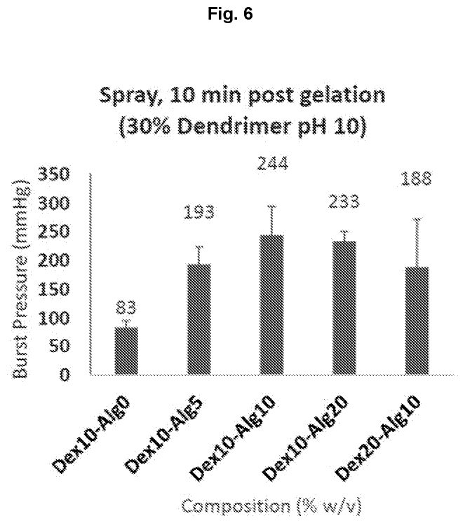

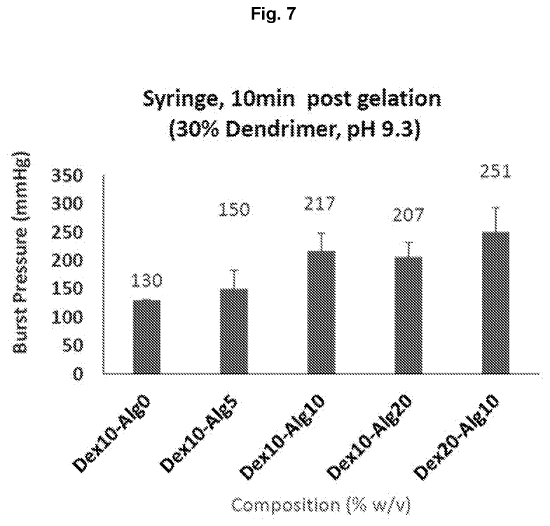

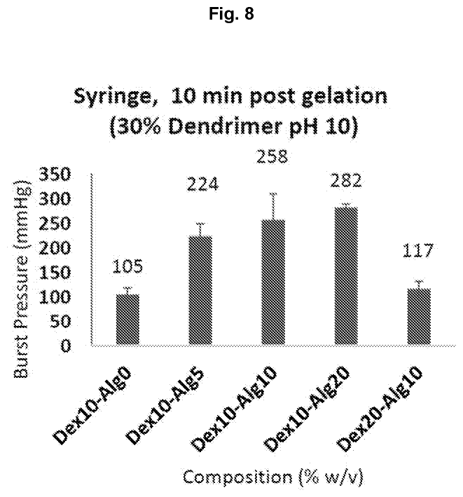

FIGS. 6, 7, and 8 depict the burst pressure results following different application methods of the material (spray vs. syringe) and the effect of the pH of the dendrimer solution on outcomes (pH=9.3 or 10).

### Example 2

### Neutral Red Uptake, Cytotoxicity Evaluation

Neutral red Uptake provides an estimation of cell viability/cytotoxicity in compliance with the FDA Guidance Document ISO-10933: “Biological Evaluation of Medical Devices”, and is extensively used for the evaluation of medical devices. Briefly, cells are exposed to culture media in which the test article has been immersed for 24 hours, any leachable or degradation product will be extracted and dissolved in the media. Cell are exposed for 24 hours to the treated media. After exposure to neutral red, viable cells are able to internalize neutral red dye and incorporate it in the lysosomes. After lysing the cells, the neutral red dye is released and can be quantified by absorption, which will be proportional to the viable cells. The percentage of viable cells is compared to a control in which cell media is not treated with any material.

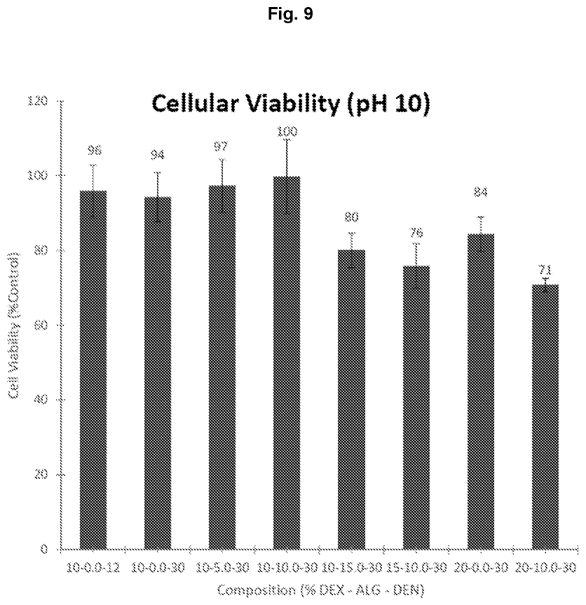

Table 1 summarizes the concentrations of the different polymers for the NRU test shown in FIG. 9. All the tested formulations exhibit viability values above 70%, which are considered biocompatible results.

TABLE 1
Test Compositions for NRU Test.

| | DextranOx (% | AlginateOx(% | Dendrimer(% |
|---|---|---|---|
| Name | w/v) | w/v) | w/v) |
| 10-0-12 | 10 | 0 | 12 |
| 12-0-30 | 12 | 0 | 30 |
| 10-5-30 | 10 | 5 | 30 |
| 10-10-30 | 10 | 10 | 30 |
| 10-15-30 | 10 | 15 | 30 |
| 15-10-30 | 15 | 10 | 30 |
| 20-0-30 | 20 | 0 | 30 |
| 20-10-30 | 10 | 10 | 30 |

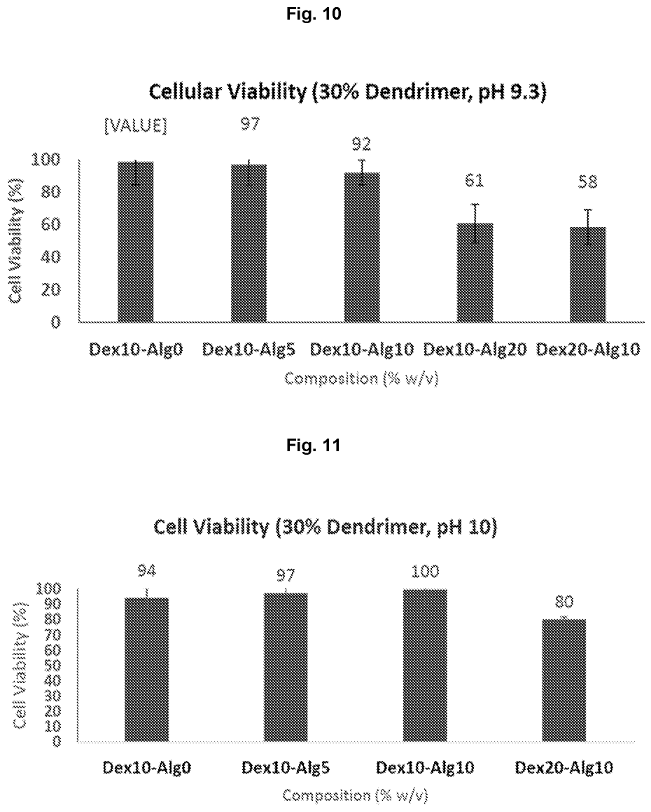

FIGS. 10 and 11 depict the effect of pH on cell viability. Most of the tested compositions maintain high cell viability, with only a small decrease in biocompatibility for some of the lower pH dendrimer samples.

### Example 3

### Exemplary Formulations of Biocompatible Adhesive

TABLE 2
Exemplary formulations

| | Den- | Den- | Oxidized | Oxidized |
|---|---|---|---|---|
| | drimer | drimer | alginate | dextran |
| Name | (wt %) | pH | (wt %) | (wt %) |
| 30% Dend pH 9.3 + 20% | 30 | 9.3 | 20 | — |
| AlgOx | | | | |
| 30% Dend pH 10.6 + 20% | 30 | 10.6 | 20 | — |
| AlgOx | | | | |
| 15% Dend pH 9.3 + 20% | 15 | 9.3 | 20 | — |
| AlgOx | | | | |
| 15% Dend pH 10.6 + 20% | 15 | 10.6 | 20 | — |
| AlgOx | | | | |
| 30% Dend pH 9.3 + 10% | 30 | 9.3 | 10 | — |
| AlgOx | | | | |
| 30% Dend pH 10.6 + 10% | 30 | 10.6 | 10 | — |
| AlgOx | | | | |
| 15% Dend pH 9.3 + 10% | 15 | 9.3 | 10 | — |
| AlgOx | | | | |
| 15% Dend pH 10.6 + 10% | 15 | 10.6 | 10 | — |
| AlgOx | | | | |
| 30% Dend pH 10.6 + 15% | 30 | 10.6 | 10 | 15 |
| DexOx & | | | | |
| 10% AlgOx | | | | |
| 30% Dend pH 9.3 + 15% | 30 | 9.3 | 10 | 15 |
| DexOx & | | | | |
| 10% AlgOx | | | | |
| 30% Dend pH 10.6 + 15% | 30 | 10.6 | 3 | 15 |
| DexOx & | | | | |
| 3% AlgOx | | | | |
| 30% Dend pH 9.3 + 15% | 30 | 9.3 | 3 | 15 |
| DexOx & | | | | |
| 3% AlgOx | | | | |

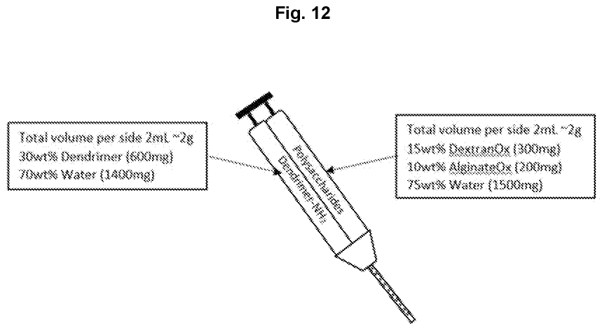

References below to “dendrimer side” and “polysaccharide side” relate to the two mixtures charged separately in a double-barrel syringe. See FIG. 12.

### 30% Dend pH 9.3+20% AlgOx

A sealant formulation where the dendrimer side contains 30 wt % in aqueous solution at pH 9.3 of PAMAM dendrimer where 25% of its surface groups are amines, and the polysaccharide side contains 20 wt % in aqueous solution of oxidized alginate where 40% of the monosaccharides contain aldehyde groups. Once the two liquid components are mixed, they form a hydrogel in about 10 seconds.

### 30% Dend pH 10.6+20% AlgOx

A sealant formulation where the dendrimer side contains 30 wt % in aqueous solution at pH 10.6 of PAMAM dendrimer where 25% of its surface groups are amines, and the polysaccharide side contains 20 wt % in aqueous solution of oxidized alginate where 40% of the monosaccharides contain aldehyde groups. Once the two liquid components are mixed, they form a hydrogel in less than 5 seconds.

### 15% Dend pH 9.3+20% AlgOx

A sealant formulation where the dendrimer side contains 15 wt % in aqueous solution at pH 9.3 of PAMAM dendrimer where 25% of its surface groups are amines, and the polysaccharide side contains 20 wt % in aqueous solution of oxidized alginate where 40% of the monosaccharides contain aldehyde groups. Once the two liquid components are mixed, they form a hydrogel in about 30 seconds.

### 15% Dend pH 10.6+20% AlgOx

A sealant formulation where the dendrimer side contains 15 wt % in aqueous solution at pH 10.6 of PAMAM dendrimer where 25% of its surface groups are amines, and the polysaccharide side contains 20 wt % in aqueous solution of oxidized alginate where 40% of the monosaccharides contain aldehyde groups. Once the two liquid components are mixed, they form a hydrogel in about 15 seconds.

### 30% Dend pH 9.3+10% AlgOx

A sealant formulation where the dendrimer side contains 30 wt % in aqueous solution at pH 9.3 of PAMAM dendrimer where 25% of its surface groups are amines, and the polysaccharide side contains 10 wt % in aqueous solution of oxidized alginate where 40% of the monosaccharides contain aldehyde groups. Once the two liquid components are mixed, they form a hydrogel in about 20 seconds.

### 30% Dend pH 10.6+10% AlgOx

A sealant formulation where the dendrimer side contains 30 wt % in aqueous solution at pH 10.6 of PAMAM dendrimer where 25% of its surface groups are amines, and the polysaccharide side contains 10 wt % in aqueous solution of oxidized alginate where 40% of the monosaccharides contain aldehyde groups. Once the two liquid components are mixed, they form a hydrogel in about 10 seconds.

### 15% Dend pH 9.3+10% AlgOx

A sealant formulation where the dendrimer side contains 15 wt % in aqueous solution at pH 9.3 of PAMAM dendrimer where 25% of its surface groups are amines, and the polysaccharide side contains 10 wt % in aqueous solution of oxidized alginate where 40% of the monosaccharides contain aldehyde groups. Once the two liquid components are mixed, they form a hydrogel in about 45 seconds.

### 15% Dend pH 10.6+10% AlgOx

A sealant formulation where the dendrimer side contains 15 wt % in aqueous solution at pH 10.6 of PAMAM dendrimer where 25% of its surface groups are amines, and the polysaccharide side contains 10 wt % in aqueous solution of oxidized alginate where 40% of the monosaccharides contain aldehyde groups. Once the two liquid components are mixed, they form a hydrogel in about 25 seconds.

### 30% Dend pH 10.6+15% DexOx & 10% AlgOx

A sealant formulation where the dendrimer side contains 30 wt % in aqueous solution at pH 10.6 of PAMAM dendrimer where 25% of its surface groups are amines, and the polysaccharide side contains 10 wt % in aqueous solution of oxidized alginate where 40% of its monomeric units contain aldehyde groups and 15 wt % of oxidized dextran where 50% of its monomeric units contain aldehyde groups. Once the two liquid components are mixed, they form a hydrogel in less than 5 seconds.

### 30% Dend pH 9.3+15% DexOx & 10% AlgOx

A sealant formulation where the dendrimer side contains 30 wt % in aqueous solution at pH 9.3 of PAMAM dendrimer where 25% of its surface groups are amines, and the polysaccharide side contains 10 wt % in aqueous solution of oxidized alginate where 40% of its monomeric units contain aldehyde groups and 15 wt % of oxidized dextran where 50% of its monomeric units contain aldehyde groups. Once the two liquid components are mixed, they form a hydrogel in about 10 seconds.

### 30% Dend pH 10.6+15% DexOx & 3% AlgOx

A sealant formulation where the dendrimer side contains 30 wt % in aqueous solution at pH 10.6 of PAMAM dendrimer where 25% of its surface groups are amines, and the polysaccharide side contains 3 wt % in aqueous solution of oxidized alginate where 40% of its monomeric units contain aldehyde groups and 15 wt % of oxidized dextran where 50% of its monomeric units contain aldehyde groups. Once the two liquid components are mixed, they form a hydrogel in less than 5 seconds.

### 30% Dend pH 9.3+15% DexOx & 3% AlgOx

A sealant formulation where the dendrimer side contains 30 wt % in aqueous solution at pH 9.3 of PAMAM dendrimer where 25% of its surface groups are amines, and the polysaccharide side contains 3 wt % in aqueous solution of oxidized alginate where 40% of its monomeric units contain aldehyde groups and 15 wt % of oxidized dextran where 50% of its monomeric units contain aldehyde groups. Once the two liquid components are mixed, they form a hydrogel in about 15 seconds.

The sealant composition is described by the wt % of each one of the components in their respective syringe sides. The example in FIG. 12 shows a syringe that contains: 30 wt % Dendrimer+15 wt % oxidized dextran and 10 wt % oxidized algiante.

The final composition of the sealant (once it is mixed and applied) would be 15 wt % Dendrimer+7.5 wt % oxidized dextran and 10 wt % oxidized algiante as the mixture is 1:1 from each side.

We claim:

1. A biocompatible adhesive material, comprising a dendrimer component, and a linear or branched polymer component.

2. The biocompatible adhesive material of claim 1, wherein the dendrimer has a molecular weight of about 1,000 to about 1,000,000 Da.

3. The biocompatible adhesive material of claim 1, wherein the dendrimer has a molecular weight of about 5,000 to about 500,000 Da.

4. The biocompatible adhesive material of claim 1, wherein the dendrimer has a molecular weight of about 10,000 to about 100,000 Da.

5. The biocompatible adhesive material of any one of claims 1-4, wherein the dendrimer is a generation dendrimer having primary amines on at least 10% of the dendrimer's surface groups.

6. The biocompatible adhesive material of any one of claims 1-4, wherein the dendrimer is a generation dendrimer having primary amines on at least 25% of the dendrimer's surface groups.

7. The biocompatible adhesive material of any one of claims 1-4, wherein the dendrimer is a generation dendrimer having primary amines on at least 40% of the dendrimer's surface groups.

8. The biocompatible adhesive material of any one of claims 1-7, wherein the dendrimer extends through at least 2-6 generations.

9. The biocompatible adhesive material of any one of claims 1-7, wherein the dendrimer extends through at least 3-5 generations.

10. The biocompatible adhesive material of any one of claims 1-7, wherein the dendrimer is a generation 5 PAMAM-derived dendrimer.

11. The biocompatible adhesive material of any one of claims 1-10, wherein the linear or branched polymer is an anionic polymer.

12. The biocompatible adhesive material of any one of claims 1-10, wherein the linear or branched polymer is a polysaccharide.

13. The biocompatible adhesive material of any one of claims 1-10, wherein the linear or branched polymer is an oxidized alginate.

14. The biocompatible adhesive material of any one of claims 1-13, wherein the linear or branched polymer has a molecular weight from about 3,000 to about 3,000,000 Da.

15. The biocompatible adhesive material of any one of claims 1-13, wherein the linear or branched polymer has a molecular weight from about 10,000 to about 1,000,000 Da.

16. The biocompatible adhesive material of any one of claims 1-13, wherein the linear or branched polymer has a molecular weight from about 30,000 to about 300,000 Da.

17. The biocompatible adhesive material of any one of claims 1-16, further comprising a second linear or branched polymer component.

18. The biocompatible adhesive material of claim 17, wherein the second linear or branched polymer is an oxidized polysaccharide.

19. The biocompatible adhesive material of claim 17, wherein the second linear or branched polymer is an oxidized dextran.

20. The biocompatible adhesive material of claim 19, wherein at least 25% of the dextran's primary hydroxyls are oxidized to aldehydes.

21. The biocompatible adhesive material of claim 19, wherein at least 50% of the dextran's primary hydroxyls are oxidized to aldehydes.

22. The biocompatible adhesive material of claim 19, wherein at least 75% of the dextran's primary hydroxyls are oxidized to aldehydes.

23. The biocompatible adhesive material of any one of claims 17-22, wherein the second linear or branched polymer component further comprises an additive selected from the group consisting of foaming agents, pH modifiers, thickeners, antimicrobial agents, colorants, surfactants, radio-opaque agents, and biologically active components.

24. The biocompatible adhesive material of any one of claims 17-23, wherein the second linear or branched polymer component is in a solution.

25. The biocompatible adhesive material of any one of claims 17-23, wherein the second linear or branched polymer component is in an aqueous solution.

26. The biocompatible adhesive material of claim 25, wherein the aqueous solution has a pH from about 7 to about 11.

27. The biocompatible adhesive material of claim 25, wherein the aqueous solution has a pH from about 8 to about 10.

28. The biocompatible adhesive material of claim 25, wherein the aqueous solution has a pH about 9.

29. The biocompatible adhesive material of any one of claims 17-28, wherein the second linear or branched polymer component constitutes 1-30 wt % of the aqueous solution.

30. The biocompatible adhesive material of any one of claims 1-29, wherein the dendrimer component or the linear or branched polymer component further comprises an additive selected from the group consisting of foaming agents, pH modifiers, thickeners, antimicrobial agents, colorants, surfactants, radio-opaque agents, and biologically active component

31. The biocompatible adhesive material of any one of claims 1-30, wherein the dendrimer component or the linear or branched polymer component is in a solution.

32. The biocompatible adhesive material of any one of claims 1-30, wherein the dendrimer component or the linear or branched polymer component is in an aqueous solution.

33. The biocompatible adhesive material of claim 32, wherein the aqueous solution has a pH from about 7 to about 11.

34. The biocompatible adhesive material of claim 32, wherein the aqueous solution has a pH from about 8 to about 10.

35. The biocompatible adhesive material of claim 32, wherein the aqueous solution has a pH about 9.

36. The biocompatible adhesive material of any one of claims 1-35, wherein the dendrimer component constitutes 1-50 wt % of the aqueous solution.

37. The biocompatible adhesive material of any one of claims 1-36, wherein the linear or branched polymer component constitutes 10-40 wt % of the aqueous solution.

38. A method of adhering, sealing, or treating one or more biological tissues or prosthetic materials, comprising applying to one or more surfaces of said one or more biological tissues or prosthetic materials a biocompatible adhesive material of any one of claims 1-37.

39. The method of claim 38, wherein the biocompatible adhesive material is applied as a spray.

40. The method of claim 38, wherein the biocompatible adhesive material is applied via a syringe.
